(19) United States
(12) Patent Application Publication
(10) Pub. No.: US 2022/0330928 A1
(43) Pub. Date: Oct. 20, 2022

(54) Multi-Orientation Razor Blade

(71) Applicant: David Alexander Phillips, Albuquerque, NM (US)

(72) Inventor: David Alexander Phillips, Albuquerque, NM (US)

(21) Appl. No.: 17/718,219

(22) Filed: Apr. 11, 2022

Related U.S. Application Data
(60) Provisional application No. 63/176,998, filed on Apr. 20, 2021.

(51) Int. Cl. A61B 10/02; B26B 21/56

(52) U.S. Cl. CPC A61B 10/0266; B26B 21/56

(57) ABSTRACT

A multi-orientation razor. The multi-orientation includes a substantially flat metal blade member being frangible about a central axis. The substantially flat metal blade member can have on a first peripheral edge thereof a first straight sharp edge. The first straight sharp edge can be parallel to the central axis. The first peripheral edge further includes a first concavely curved sharp edge. The substantially flat metal blade member can have on a second peripheral edge thereof a second straight sharp edge. The second straight sharp edge can be parallel to the central axis. The second peripheral edge can further include a second concavely curved sharp edge.

### TECHNICAL FIELD

The present disclosure relates to razors suitable for shave biopsies.

### BACKGROUND

When performing shave biopsies for cutaneous/skin lesions, the final cut of the biopsy completely separating the lesion from the skin is often difficult to complete. One reason for the difficulty is because when using biopsy razors, the lesion will “flip over” on a final stalk of skin and off of the razor, remaining attached to the skin.

An additional problem that can arise, particularly when attempting to avoid letting the skin lesion “flip over” and off the razor, is to accidentally continue the cut of the biopsy beyond the intended margin at the end of the cut due to difficulty in completing the final cut. In some cases, a surgeon doing the biopsy must grasp the lesion with forceps, or more commonly, use the back of a cotton-tipped applicator to hold the lesion on top of the razor, and prevent it from flipping over. Often this must be done by an assistant as the surgeon is using one hand to manipulate the razor and the other to stabilize/elevate the skin from which the biopsy is being taken.

### BRIEF DESCRIPTION OF THE DRAWINGS

The following detailed description of embodiments of the present disclosure can be best understood when read in conjunction with the drawings enclosed herewith:

FIG. 1 is a plan view of a double-edged razor blade;

FIG. 2 is perspective view of the portion of the double-edged razor blade of FIG. 1 being used to perform a biopsy;

FIG. 3 is a plan view of a sing-edged razor blade;

FIG. 4 is perspective view of the portion of the single-edged razor blade of FIG. 3 being used to perform a biopsy;

FIG. 5 is a plan view of a multi-orientation razor blade;

FIG. 6 is perspective view of the portion of the multi-orientation razor blade of FIG. 5 being used to perform a biopsy;

FIG. 7 is a plan view of a multi-orientation razor blade;

FIG. 8 is a plan view of a multi-orientation razor blade;

FIG. 9 is a plan view of a multi-orientation razor blade;

FIG. 10 is a plan view of a multi-orientation razor blade;

FIG. 11 is a plan view of a multi-orientation razor blade;

FIG. 12 is a plan view of a multi-orientation razor blade;

FIG. 13 is a plan view of a multi-orientation razor blade;

FIG. 14 is a perspective view of a multi-orientation razor blade;

FIG. 15 is a perspective view of a multi-orientation razor blade;

FIG. 16 is a perspective view of a multi-orientation razor blade;

FIG. 17 is a perspective view of a multi-orientation razor blade; and

FIG. 18 is a diagram showing representative features and dimensions for a multi-orientation razor blade.

The embodiments set forth in the drawings are illustrative in nature and not intended to be limiting. Moreover, individual features of the drawings and the disclosure will be more fully apparent and understood in view of the detailed description.

### DETAILED DESCRIPTION

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the apparatuses, devices, systems, and methods described herein. None of the features or components shown in the drawings or discussed below should be taken as mandatory for any specific implementation of any of these the apparatuses, devices, systems, or methods unless specifically designated as mandatory. For ease of reading and clarity, certain components, modules, or methods may be described solely in connection with a specific FIG. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

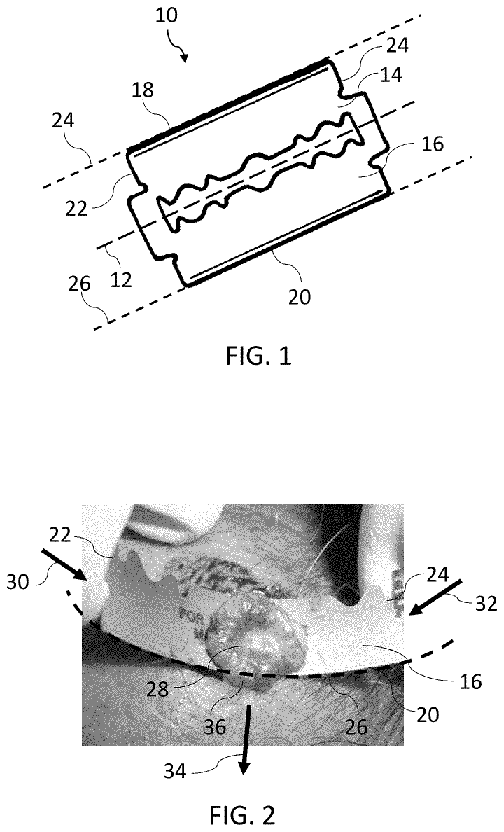

Referring to FIG. 1, there is shown a double-edged razor blade 10 of the type commonly used to perform skin biopsies. The double-edged razor blade 10 is made of metal with sharpened edges and is generally thin and flat in an imaginary plane of the razor, which for illustration purposes is a plane parallel with the plane of the paper of FIG. 1. The double-edged razor blade 10 is symmetrical about a central axis 12, and can be physically broken for use along the central axis 12 to provide for two single-edged razor blades, termed herein as a first razor blade 14 and a second razor blade 16. The sharp edges of the double-edged razor blade 10 are straight. That is, for example, the first sharp edge 18 of the first razor blade 14 is straight from a first side 22 of the double-edged razor blade 10 to a second side 24 of the razor blade 10, following the path of a first imaginary straight line 24. Likewise, the second sharp edge 20 of the second razor blade 16 is straight from a first side 22 of the double-edged razor blade 10 to a second side 24 of the second razor blade 16, following the path of a second imaginary straight line 26. Additionally, the first sharp edge 18 and the second sharp edge 20 are generally parallel and co-planar, residing in the imaginary plane of the razor. Further, the first sharp edge 18 and the second sharp edge 20 are generally parallel and co-planar with the central axis 12, residing in the imaginary plane of the razor.

Referring to FIG. 2, there is shown a razor being used for a biopsy. As shown, a razor of the type shown in FIG. 1 is being used to remove a skin lesion 30 in a shave biopsy. As shown, the second razor 16, which is a broken-off half of the double-edged razor blade 10, is squeezed by a user's fingers at the first side 22 and second side 24 in the direction of the arrows 30 and 32, respectively, to produce a slight bow in the second sharp edge 20, causing a curvature of the imaginary plane of the razor, and a corresponding curvature of the path of the second imaginary straight line 26. The first razor 16 is moved in the direction of arrow 34 to force the second sharp edge 20 under and through the skin lesion 28. At the stage of removal shown in FIG. 2, a final stalk of skin 36 remains un-severed. It at this stage that the cut portion of the skin lesion 28 must be stabilized to prevent loss of control of its position. The skin lesion at this stage of removal can “flip over” or otherwise slip off the second sharp edge 20, so that the surgeon doing the biopsy must stabilize the lesion with forceps, or more commonly, use the back of a cotton-tipped applicator to hold the lesion on top of the razor, and prevent it from flipping over.

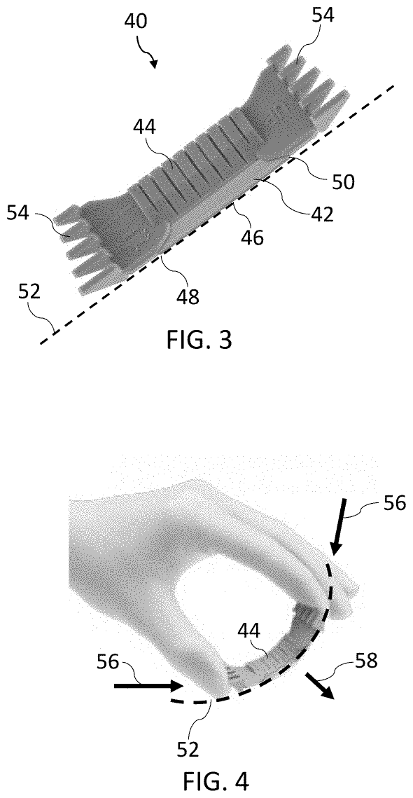

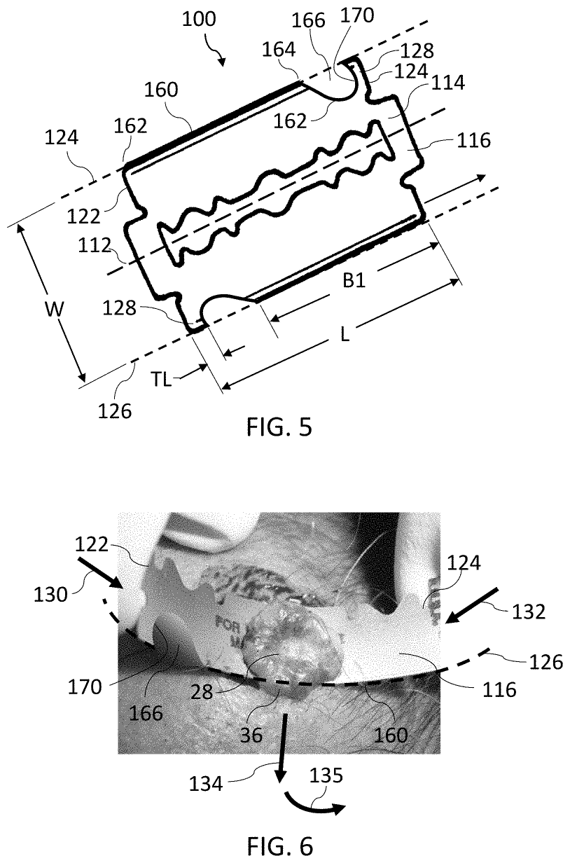

Other special-purpose configurations of razors having straight sharpened edges are available. For example, as depicted in FIGS. 3 and 4, a single-edged razor 40 can have a blade 42 embedded in a molded plastic flexible housing 44, which can be gripped for use as described above with respect to either of the first razor blade 14 and the second razor blade 16 of the double-edged razor blade 10. The sharp edge 46 of the single-edged razor blade 40 is straight. That is, the sharp edge 46 of the single-edged razor blade 40 is straight from a first side 48 of the sharp edge 46 to a second side 50 of the sharp edge 46, following the path of an imaginary straight line 52. In an embodiment, single-edged razor 40 can have at each end thereof finger grips 54. As shown in FIG. 5, a user, such as a surgeon, can press inwardly in the direction of arrows 56 to produce a slight bow in the sharp edge 46, causing a curvature of the imaginary straight line 52. The user can then move the single-edged razor 40 in the direction of the arrow 58 to remove a skin lesion, as described above. The same issues with controlling the partially removed skin lesion occur with the special-purpose single-edged razor 40, as described above for the double-edged razor blade 10.

Referring to FIG. 5, there is shown a multi-orientation razor 100 useful for performing skin biopsies. The multi-orientation razor 100 can be made of metal with sharpened edges and is generally thin and flat in an imaginary plane of the razor, which for illustration purposes is a plane parallel to the plane of the paper of FIG. 5. The multi-orientation razor 100 can be symmetrical about a central axis 112 and can be physically broken for use along the central axis 112 to provide for a first multi-orientation razor blade 114 and a second multi-orientation razor blade 116.

The term “multi-orientation” as used herein refers to the orientation of a sharp edge of a razor having multiple blade orientations, for example in relation to a central axis, such as central axis 112, in the plane of the razor. In an embodiment, each multi-orientation razor blade has two blade components, a first straight component, the straight component having a sharp edge oriented at a fixed angle with respect to the central axis, and a second curved component, the curved component having a sharp edge oriented at a variable angle, the angle of the curved component changing along the curvature of the second curved component, with respect to the central axis.

Referring again to FIGS. 5 and 6, the two blade components will be described with respect to the first multi-orientation razor blade 114, and it is understood that the description can be likewise applied to the second multi-orientation razor blade 116, but for clarity and brevity is not repeated in full. The first multi-orientation razor blade 114 has a first straight blade component 160 and a second curved blade component 162. The first straight blade component 160 is straight from a first end 162 near the first side 122 of the razor blade 100 and lies on a path of a first imaginary straight line 124 to a second end 164 disposed intermediate between the first side 122 and a second side 124. The first imaginary straight line 124 can be parallel and co-planar with the central axis 112. The second end 166 can be a transition point at which the second blade component 162 begins and continues in a concave curvilinear path defining a notch member 166 having an opening spanning at least a portion of the distance between the second end 164 and the second side 124. The notch member 166 is disposed interior to boundaries formed by the first imaginary straight line 124 and the central axis 112. That is, in this embodiment, no portion of the first multi-orientation razor blade 114 of the multi-orientation razor 100 extends beyond the boundary of the first imaginary straight line 124, which is co-linear with the first straight blade component 160. The second blade component can be concave with respect to the first imaginary straight line 124 corresponding to the location of the first blade component of the first multi-orientation blade 114.

The features of the first multi-orientation razor blade 114 can be duplicated or mirrored in the second multi-orientation razor blade 116. At least portions (including all in some embodiments) of the first straight blade component 160 and the second curved blade component 166 are each sharpened to razor sharpness sufficient to be used for skin biopsies. In an embodiment, the first straight component 160 and the second curved component 162 can, together, comprise a continuous razor-sharp edge. In an embodiment, the second curved component 162 can be partially razor sharpened. For example, in an embodiment, only a portion of the concave curvilinear path of the notch portion 166 is razor sharp, for example, the edge portion 170 nearest to the second edge 124.

As depicted in FIG. 5, and in respect to the second multi-orientation razor blade 116, but applicable also the first multi-orientation razor blade 114, certain dimensions can be described. A blade length L dimension, measured as the distance between the first side 122 and the second side 124 of the multi-orientation razor blade 100 can from about 25 mm to about 50 mm, and can be 43 mm (1 11/16 in). The first blade length B1 dimension of the first straight blade component 160 can be from about 50% to about 80% of the blade length L. In an embodiment, the first blade length B1 can be about 50%, 60%, 70%, 75%, or 80% of the blade length L. A tab length TL dimension of a tab 128 measured along a path corresponding to either of the first imaginary straight line 124 or the second imaginary straight line 126, and spanning from the first side 122 or the second side 124 to a transition location to the opening of the notch 166 can be about 5%, 10%, 15%, 20%, 25%, or 30% of the blade length L. The blade width W dimension of the multi-orientation razor 100 can be from about 15 mm to about 30 mm. In an embodiment, blade width W can be 22 mm (⅞ in). The shape of the notch 166 can be varied as desired, including in the opening dimension represented in FIG. 5 by B1−TL, as well as the depth of the notch as measured off of one of either the first imaginary straight line 124 or the second imaginary line 126.

Referring now to FIG. 6, the advantageous benefits of a multi-orientation razor blade, such as the multi-orientation razor blade 100, is shown. As shown, the second multi-orientation razor blade 116 is being used to remove a skin lesion 28 in a shave biopsy. As shown, the second multi-orientation razor blade 116, which is a broken-off half of the multi-orientation razor blade 100, is squeezed by a user's fingers at the first side 122 and second side 124 in the direction of the arrows 130 and 132, respectively, to produce a slight bow in the first sharp edge 160, causing a curvature of the imaginary plane of the razor, and a corresponding curvature of the path of the second imaginary straight line 126. The second multi-orientation razor blade 116 is moved in the direction of arrow 134 to force the first sharp edge 160 under and through the skin lesion 28. At the stage of removal wherein a final stalk of skin 36 remains un-severed, the second multi-orientation razor blade 116 is moved in the direction of arrow 135, thereby sliding the second multi-orientation razor blade 116 laterally until the second sharp edge 170 engages and cuts the final stalk of skin 36. In an example, the second multi-orientation razor blade 116 is moved by a surgeon such that the notch 166 engages the final stalk of skin 36 and the second sharp edge 170 cuts the final stalk of skin 36. As can be understood, the skin lesion 28 can be removed with a single, smooth motion of the second multi-orientation razor blade 116 by providing for two different angles of engagement between the second multi-orientation razor blade 116 and the skin lesion. The resulting removal of a skin lesion 28 can be accomplished without the need for any additional stabilizing of the skin lesion 28. The first sharp edge 160 cuts in the traditional manner of blades of the type shown in FIG. 1, while the second sharp edge 170, oriented differently with respect to, for example, the centerline 112, can be used to cut the final stalk of skin 36.

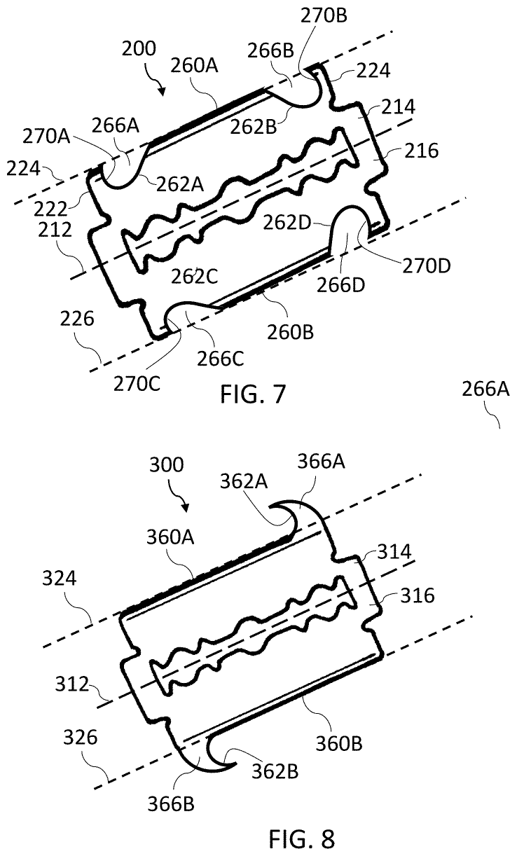

Referring now to FIG. 7, there is illustrated another example of a multi-orientation razor, shown as multi-orientation razor 200. The multi-orientation razor 200 can have every structure, function, and benefit as described above with respect to the multi-orientation razor 100 of FIG. 5, with one difference being the presence of more than one notch, and corresponding second sharp edge, on each of the blade sides of the multi-orientation razor 200. The multi-orientation razor 200 can be substantially symmetrical about a central axis 212 and can be physically broken for use along the central axis 212 to provide for a first multi-orientation razor blade 214 and a second multi-orientation razor blade 216. The first multi-orientation razor blade 214 has a first straight blade component 260A and two second curved blade components 262A and 262B. The first straight blade component 260A is straight and extends between the two second curved blade components 262A and 262B and lies on a path of a first imaginary straight line 224. The first imaginary straight line 224 is substantially parallel and co-planar with the central axis 212. The first straight blade component 260A transitions to each of the two second curved blade components 262A and 262B and continues in a concave curvilinear path defining the notch members 266A and 266B, respectively. Each of the notches has an opening spanning at least a portion of the distance between the ends of the first blade component 260A and the first side 222 and the second side 224. One or both of the notch member 266A and the notch member 266B is disposed interior to boundaries formed by the first imaginary straight line 224 and the central axis 212. That is, in this embodiment, no portion of the first multi-orientation razor blade 214 of the multi-orientation razor 200 extends beyond the boundary of the first imaginary straight line 224, which is co-linear with the first straight blade component 260. The two second curved blade components 262A and 262B can each be concave with respect to the first imaginary straight line 224 corresponding to the location of the first blade component 260A of the first multi-orientation blade 214.

Similarly, the second multi-orientation razor blade 216 has a second straight blade component 260B and two second curved blade components 262C and 262D. The first straight blade component 260A is straight and extends between the two second curved blade components 262A and 262B and lies on a path of a second imaginary straight line 226. The second imaginary straight line 226 is substantially parallel and co-planar with the central axis 212. The second straight blade component 260B transitions to each of the two second curved blade components 262C and 262D and continues in a concave curvilinear path defining the notch members 266C and 266D, respectively. Each of the notch members has an opening spanning at least a portion of the distance between the ends of the second blade component 260B and the first side 222 and the second side 224. One or both of the notch member 266C and the notch member 266D is disposed interior to boundaries formed by the second imaginary straight line 226 and the central axis 212. That is, in this embodiment, no portion of the second multi-orientation razor blade 216 of the multi-orientation razor 200 extends beyond the boundary of the second imaginary straight line 226, which is co-linear with the second straight blade component 260B. The two second curved blade components 262C and 262D can each be concave with respect to the second imaginary straight line 226 corresponding to the location of the second blade component 260B of the second multi-orientation blade 214.

Referring now to FIG. 8, there is illustrated another example of a multi-orientation razor, shown as multi-orientation razor 300. The multi-orientation razor 300 can have the structure, function, and benefits of the previously described razors, with the differences and distinctions as described herein. In general, the multi-orientation razor 300 differs from previously described razors in that the second blade component extends outside the boundary of the imaginary straight lines corresponding to the position and orientation of the first blade component. The multi-orientation razor 300 can be substantially symmetrical about a central axis 312 and can be physically broken for use along the central axis 312 to provide for a first multi-orientation razor blade 314 and a second multi-orientation razor blade 316. The first multi-orientation razor blade 314 has a first straight blade component 360A and a second curved blade component 362A. The first straight blade component 360A is straight and lies on a path of a first imaginary straight line 324. The first imaginary straight line 324 is substantially parallel and co-planar with the central axis 312. The first straight blade component 360A transitions in a concave curvilinear path defining the second curved blade component 362A being the concave edge of a second blade tab 366A, which can extend in a hook-like shape, disposed distally beyond the first imaginary straight line 324 aligned with the first blade component 360A. In an embodiment, the shape of the second blade tab 366A can be described as “shark tooth-shaped, triangular-shaped, hook-shaped, and the like.

Likewise, the second multi-orientation razor blade 316 has a third straight blade component 360B and a fourth curved blade component 362B. The third straight blade component 360B is straight and lies on a path of a second imaginary straight line 326. The second imaginary straight line 326 is substantially parallel and co-planar with the central axis 312. The third straight blade component 360B transitions in a concave curvilinear path defining the fourth curved blade component 362B being the concave edge of a second blade tab 366A, which can extend in a hook-like shape, disposed distally beyond the second imaginary straight line 326 aligned with the third blade component 360B. In an embodiment, the shape of the second blade tab 366A can be described as “shark tooth-shaped, triangular-shaped, hook-shaped, and the like.

In an embodiment, the multi-orientation razor 300 as depicted in FIG. 8 can further comprise one or more additional second blade tabs. For example, as the straight blade components of the multi-orientation razor 200 of FIG. 7 are bounded on each end by notches, the first straight blade component 360A and/or the third straight blade component 360B can be bounded by second blade tabs, such that the multi-orientation razor 300 can have three or four second blade tabs.

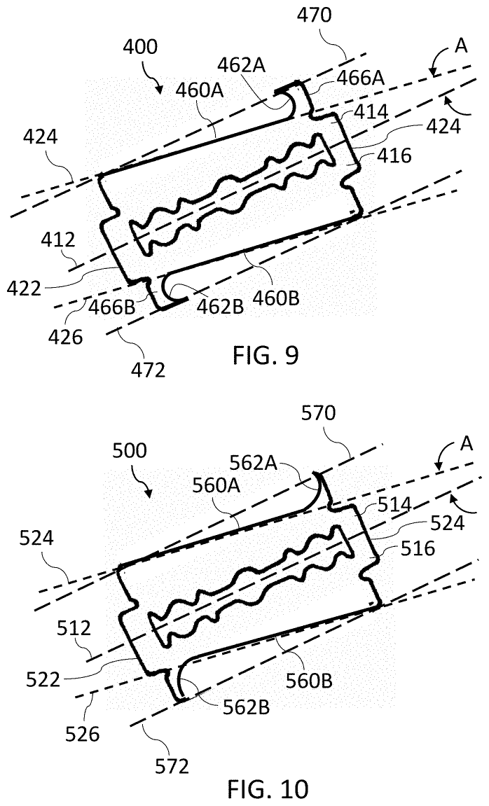

Referring now to FIG. 9, there is illustrated another example of a multi-orientation razor, shown as multi-orientation razor 400. The multi-orientation razor 400 can have the structure, function, and benefits of the previously described razors, with the differences and distinctions as described herein. In general, the multi-orientation razor 400 differs from previously described razors in that the first blade component extends on an imaginary straight line corresponding to the position and orientation of the first blade component, and wherein the imaginary straight line of the first blade component is not parallel to the central axis. The multi-orientation razor 400 can be substantially symmetrical about a central axis 412 and can be physically broken for use along the central axis 412 to provide for a first multi-orientation razor blade 414 and a second multi-orientation razor blade 416. The first multi-orientation razor blade 414 has a first straight blade component 460A and a second curved blade component 462A. The first straight blade component 460A is straight and lies on a path of a first imaginary straight line 424. The first imaginary straight line 424 is not parallel with the central axis 412. The first straight blade component 460A transitions in a concave curvilinear path defining the second curved blade component 462A being the substantially semi-circular concave edge of a second blade tab 466A, which can extend in a hook-like shape, disposed distally beyond the first imaginary straight line 424 aligned with the first blade component 460A.

Likewise, the second multi-orientation razor blade 416 has a third straight blade component 460B and a fourth curved blade component 462B. The third straight blade component 460B is straight and lies on a path of a second imaginary straight line 426. The second imaginary straight line 426 is not parallel with the central axis 412, but lies at an angle A thereto, the angle A being between about 5 degrees and about 45 degrees, or between about 10 degrees and about 30 degrees. The third straight blade component 360B transitions in a concave curvilinear path defining the fourth curved blade component 462B being the substantially semi-circular concave edge of a second blade tab 466A, which can extend in a hook-like shape, disposed distally beyond the second imaginary straight line 326 aligned with the third blade component 460B.

In an embodiment, a third imaginary straight line 470 and a fourth imaginary straight line 472 can each be parallel to the central axis 412 and bound the limits of the structure of the multi-orientation razor 400. That is, in an embodiment, the multi-orientation razor 400 can be manufactured from a rectangular-shaped blank of material, with the rectangle being defined by the two sides 422 and 424 and the third imaginary straight line 470 and the fourth imaginary straight line 472.

Referring now to FIG. 10, there is illustrated another example of a multi-orientation razor, shown as multi-orientation razor 500. The multi-orientation razor 500 can have the structure, function, and benefits of the previously described razors, with the differences and distinctions as described herein. In general, the multi-orientation razor 500 differs from previously described multi-orientation razor 400 in that the second blade component is shaped to have a curved blade surface does not make a semi-circular shape. The multi-orientation razor 500 can be substantially symmetrical about a central axis 512 and can be physically broken for use along the central axis 512 to provide for a first multi-orientation razor blade 514 and a second multi-orientation razor blade 516. The first multi-orientation razor blade 514 has a first straight blade component 560A and a second curved blade component 562A. The first straight blade component 560A is straight and lies on a path of a first imaginary straight line 524. The first imaginary straight line 524 is not parallel with the central axis 512. The first straight blade component 560A transitions in a concave curvilinear path defining the second curved blade component 562A being the substantially quarter-circular concave edge of a second blade tab 566A, which can extend in a shark tooth-like shape, disposed distally beyond the first imaginary straight line 524 aligned with the first blade component 560A.

Likewise, the second multi-orientation razor blade 516 has a third straight blade component 560B and a fourth curved blade component 562B. The third straight blade component 560B is straight and lies on a path of a second imaginary straight line 526. The second imaginary straight line 526 is not parallel with the central axis 512, but lies at an angle A thereto, the angle A being between about 5 degrees and about 45 degrees, or between about 10 degrees and about 30 degrees. The third straight blade component 560B transitions in a concave curvilinear path defining the fourth curved blade component 562B being the substantially quarter-circular concave edge of a second blade tab 566A, which can extend in a shark tooth-like shape, disposed distally beyond the second imaginary straight line 526 aligned with the third blade component 560B.

In an embodiment, a third imaginary straight line 570 and a fourth imaginary straight line 572 can each be parallel to the central axis 512 and bound the limits of the structure of the multi-orientation razor 500. That is, in an embodiment, the multi-orientation razor 500 can be manufactured from a rectangular-shaped blank of material, with the rectangle being defined by the two sides 522 and 524 and the third imaginary straight line 570 and the fourth imaginary straight line 572.

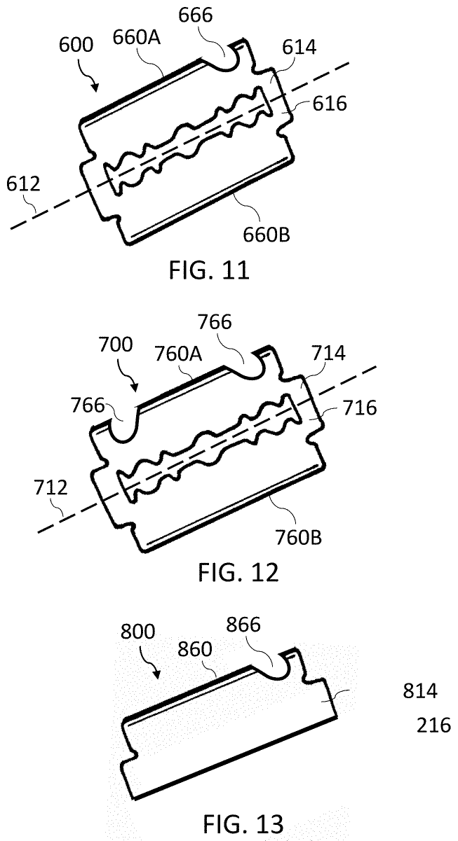

Referring now to FIGS. 11-13, certain non-limiting examples of variations and modifications of a multi-orientation razor are described. FIG. 11 shows a multi-orientation razor 600 that is not symmetrical about a central axis 612. The multi-orientation razor 600 can have a first side 614 having a straight blade component 660A and a curved blade component 666 which can be a notch as shown, or a tab, as described in any of the embodiments herein. The multi-orientation razor 600 can have a second side 616 having only a straight blade component 660B. In a similar manner, the multi-orientation razor 700 of FIG. 12 is asymmetrical about centerline 712 and can have two curved blade components 766 and a straight blade component 760A on a first side 714. The multi-orientation razor 700 can have a second side 716 having only a straight blade component 760B. In any of the embodiments described herein as having two halves, one or the other half can be supplied as a single-edged razor, such as the single-edged razor 800 shown in FIG. 13. The single-edged razor 800 can have a straight blade component 860 and a curved blade component 866 which can be a notch as shown, or a tab, as described in any of the embodiments herein.

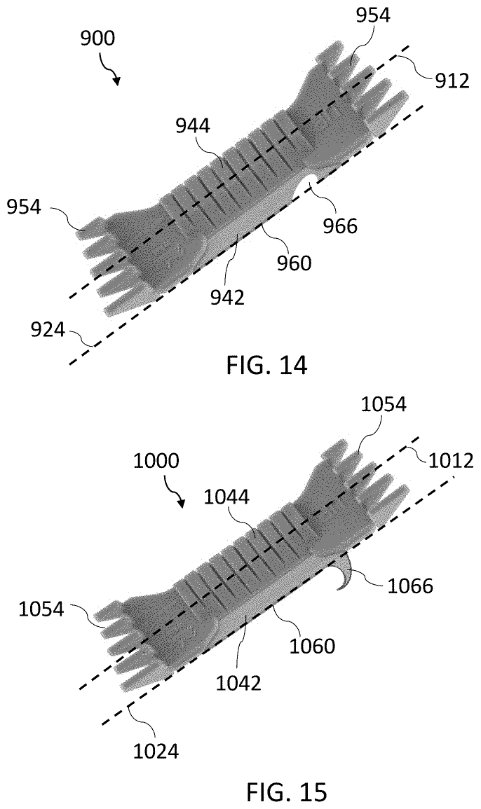

Referring now to FIGS. 14 and 15, certain non-limiting examples of variations and modifications of a multi-orientation razor are described. Specifically, the structures described in FIGS. 14 and 15 are modified versions of the razor described with respect to FIG. 3. FIG. 14 shows a single-edged razor 900 can have a blade 942 embedded in a molded plastic flexible housing 944, having a longitudinally oriented central axis 912 and finger grips 954 at each end thereof. The blade 942 comprises a straight blade component 960 that is aligned with an imaginary line 924 that is parallel and offset from the central axis 912. That is, the sharp edge of the straight blade component 960 follows the path of an imaginary straight line 924. The blade 942 comprises a curved blade component 966, indicated in FIG. 14 as a notch 966, and which can be any of the above-described curved blade components, including a concavely curved blade that is disposed interiorly to the area bounded by the central axis 912 and the imaginary line 924.

FIG. 15 shows a single-edged razor 1000 can have a blade 1042 embedded in a molded plastic flexible housing 1044, having a longitudinally oriented central axis 1012 and finger grips 1054 at each end thereof. The blade 1042 comprises a straight blade component 1060 that is aligned with an imaginary line 1024 that is parallel and offset from the central axis 1012. That is, the sharp edge of the straight blade component 1060 follows the path of an imaginary straight line 1024. The blade 1042 comprises a curved blade component 1066, indicated in FIG. 15 as a tab 1066, and which can be any of the above-described curved blade components, including a concavely curved blade that is disposed exteriorly to the area bounded by the central axis 912 and the imaginary line 924.

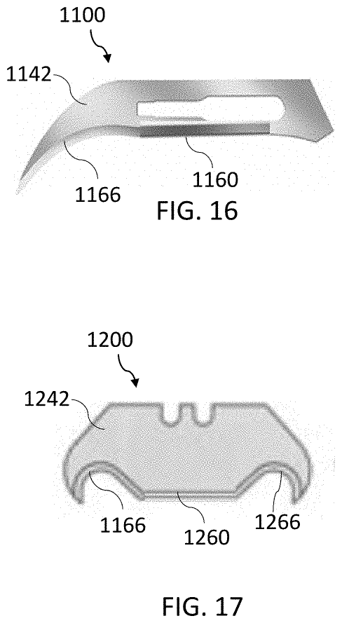

Referring now to FIGS. 16 and 17, certain non-limiting examples of a multi-orientation razor are described. FIG. 14 shows a surgical razor 1100 comprising a blade 1142 The blade 1142 comprises a straight blade component 1160 and a concavely curved blade component 1166. In an embodiment, the straight blade component 1160 and the concavely curved blade component 1166 are a continuous sharp edge. FIG. 15 shows a razor 1200 comprising a blade 1242 The blade 1242 comprises a straight blade component 1260 bounded on one or both ends by a concavely curved blade component 1266. In an embodiment, the straight blade component 1260 and the concavely curved blade component(s) 1266 are a continuous sharp edge.

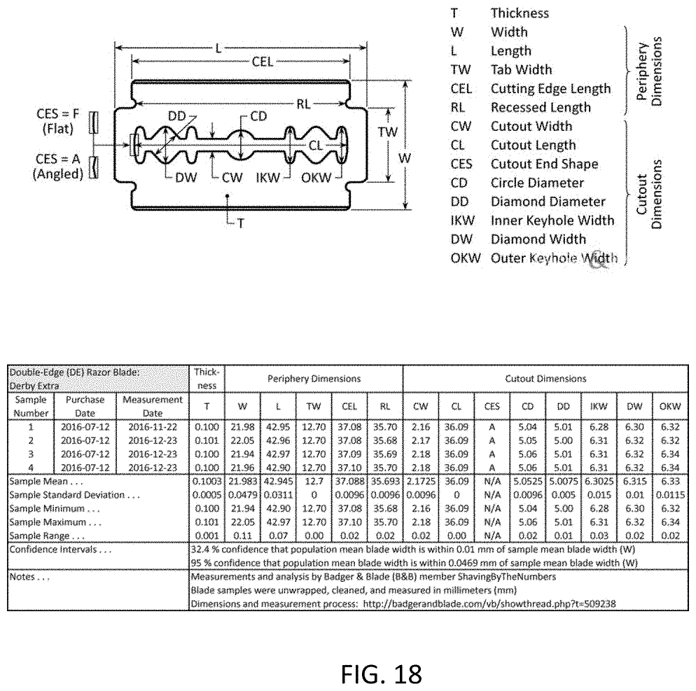

FIG. 18 shows representative features and dimensions of blades of the type described herein. In general, the dimensions can be utilized for any of the features described herein in manufacturing a multi-orientation razor. The illustrated diagrams are from Badger and Blade: http://badgerandblade.com/.

The multi-orientation razors of the present disclosure can be ISO compliant, sterile, and clean room certified. The multi-orientation razor blades can be made of stainless steel and can have a coating on the steel blade itself. Tungsten and titanium can be used as coating metals. The stainless steel can have a composition of chromium between 12% and 14.5%, a carbon content of approximately 0.6%, and the remainder iron and trace elements. However, depending on cost, variants of material composition can be used. The steel can then be crafted into long strips. The long strips of steel, being in essence a long steel belt, can be put a through a high-speed machine which punches the blade to shape. The steel blade can then be heated to temperatures of 1,075° C.-1,120° C., then quickly cooled to between −60° C. and −80° C. to harden it, before being tempered again to a temperature of 250° C.-400° C. The steel blade can then go through an etching machine where each blade is branded. A protective coating can then be bonded to the cutting edge in a process called sintering.

It is noted that terms like “specifically,” “preferably,” “commonly,” and “typically” are not utilized herein to limit the scope of the claimed disclosure or to imply that certain features are critical, essential, or even important to the structure or function of the claimed disclosure. Rather, these terms are merely intended to highlight alternative or additional features that may or may not be utilized in a particular embodiment of the present disclosure. It is also noted that terms like “substantially” and “about” are utilized herein to represent the inherent degree of uncertainty that may be attributed to any quantitative comparison, value, measurement, or other representation.

Having described the disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

All documents cited in the Detailed Description of the Disclosure are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by reference, the meaning or definition assigned to the term in this written document shall govern.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

1. A multi-orientation razor, comprising:
a substantially flat metal blade member being frangible about a central axis,
the substantially flat metal blade member having on a first peripheral edge thereof a first straight sharp edge, the first straight sharp edge being parallel to the central axis, the first peripheral edge further comprising a first concavely curved sharp edge; and
the substantially flat metal blade member having on a second peripheral edge thereof a second straight sharp edge, the second straight sharp edge being parallel to the central axis, the second peripheral edge further comprising a second concavely curved sharp edge.

2. The multi-orientation razor of claim 1, wherein the first straight sharp edge and the first concavely curved sharp edge comprise a continuous razor edge.

3. The multi-orientation razor of claim 1, wherein the substantially flat metal blade member has a blade length dimension measured parallel to the central axis and the first straight sharp edge extends a distance equal to from 50% to 90% of the blade length dimension.

4. The multi-orientation razor of claim 1, wherein the first concavely curved sharp edge defines a first notch in the first peripheral edge and the second concavely curved sharp edge defines a second notch in the second peripheral edge.

5. The multi-orientation razor of claim 1, wherein the first concavely curved sharp edge defines a first tab extending from the first peripheral edge and the second concavely curved sharp edge defines a second tab extending from the second peripheral edge.

6. The multi-orientation razor of claim 1, wherein the substantially flat metal blade member comprises stainless steel.

7. A multi-orientation razor, comprising:
a flexible metal blade member being frangible about a central axis,
the flexible metal blade member having on a first peripheral edge thereof a first straight sharp edge, the first straight sharp edge being non-parallel to the central axis, the first peripheral edge further comprising a first concavely curved sharp edge; and
the flexible metal blade member having on a second peripheral edge thereof a second straight sharp edge, the second straight sharp edge being non-parallel to the central axis, the second peripheral edge further comprising a second concavely curved sharp edge.

8. The multi-orientation razor of claim 7, wherein the first straight sharp edge and the first concavely curved sharp edge comprise a continuous razor edge.

9. The multi-orientation razor of claim 7, wherein the flexible metal blade member has a blade length dimension measured parallel to the central axis and the first straight sharp edge extends a distance equal to from 50% to 90% of the blade length dimension.

10. The multi-orientation razor of claim 6, wherein the first concavely curved sharp edge defines a first tab extending from the first peripheral edge and the second concavely curved sharp edge defines a second tab extending from the second peripheral edge.

11. The multi-orientation razor of claim 6, wherein one of the first tab and the second tab define a shark tooth shape.

12. The multi-orientation razor of claim 7, wherein an imaginary line aligned with one of the first straight sharp edge and the second straight sharp edge intersects with an imaginary line aligned with the central axis at an angle of between about 10 degrees and about 40 degrees.

13. The multi-orientation razor of claim 7, wherein the flexible metal blade member comprises stainless steel.

14. A multi-orientation razor, comprising:
a flexible metal blade member partially embedded in a molded plastic flexible housing, the molded plastic flexible housing having a longitudinally oriented central axis and finger grips at each end thereof, the flexible metal blade member having exposed on a first peripheral edge thereof a straight sharp edge, the straight sharp edge comprising a razor blade component that is aligned with a first imaginary line that is parallel and offset from a second imaginary line aligned with the central axis, the flexible metal blade member also having exposed on the first peripheral edge thereof a concavely curved blade component.

15. The multi-orientation razor of claim 14, wherein the concavely curved blade component defines a notch in the flexible metal blade member.

16. The multi-orientation razor of claim 14, wherein the concavely curved blade component defines a tab extending from the flexible metal blade member.

17. The multi-orientation razor of claim 14, wherein the flexible metal blade member comprises stainless steel.
